SEQUENCE LISTING (4 sequences)

SEQ ID NO: 1 - protein, 157 aa, Homo sapiens
MATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH

SEQ ID NO: 2 - DNA, 726 bp, Homo sapiens
  CDS (156)..(662)
ggaggtaaaggccgcgcttgggtgtccctgggtggtcgggtccccgagttgggaggggcggaaggctgaacctccagcttgagccggacaagccgattcccagcgttgagagggtagagatgaactgtgtgtgaggccaaactggatcggtcaacatggtcttccccctccccactccccaggagcccatcatggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattgacttcttccccccatcctcagacattaaagagcctgaaaaaaaaaaaaaaaaaaaaaaaaaa

SEQ ID NO: 3 - protein, 169 aa, Homo sapiens
MVFPLPTPQEPIMATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH

SEQ ID NO: 4 - DNA, 553 bp, Homo sapiens
ctgaggagcccatcatggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattgacttcttccccccatcctcagacattaaagagcctgaatgcctttgaaaaaaaaaaaaaaaaaaaa

(19) United States
(12) Patent Application Publication
(10) Pub. No.: US 2003/0092054 A1
(43) Pub. Date: May 15, 2003

(54) Protein complexes and assays for screening anti-cancer agents

(76) Inventors: Matthias Georg Christian Gstaiger, Basel (CH); Wilhelm Krek, Riehen (CH)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/276,013

(22) PCT Filed: May 11, 2001
(86) PCT No.: PCT/EP01/05403

(30) Foreign Application Priority Data
May 12, 2000 (GB) 0011439.7

(51) Int. Cl. C12Q 1/68; G01N 33/574; C12N 9/22

(52) U.S. Cl. 435/6; 435/7.23; 435/199

(57) ABSTRACT

Protein complexes containing a SKP2 binding protein (STAP1) are provided useful in identifying anti-cancer agents. Also provided are agents identified using the methods for use as therapeutics.

The invention relates to the field of cancer diagnosis and therapy. The invention also relates to the screening of compounds for potential anti-cancer activity, whether prophylactic or therapeutic. The screening assays concerned are those which seek to mimic a part of the biochemical machinery of intact cells in vivo involved in processes of cell division, gene expression and transformation which gives rise to cancers.

In the more affluent countries of the world cancer is the cause of death of roughly one person in five. The American Cancer Society in 1993 reported that the five most common cancers are those of the lung, stomach, breast, colon/rectum and the uterine cervix. Cancer is not fatal in every case and only about half the number of people who develop cancer die of it. The problem facing cancer patients and their physicians is that seeking to cure cancer is like trying to get rid of weeds. Although cancer cells can be removed surgically or destroyed with toxic compounds or with radiation, it is very hard to eliminate all of the cancerous cells. A general goal is to find better ways of selectively killing cancer cells whilst leaving normal cells of the body unaffected. Part of that effort involves identifying new anti-cancer agents.

Cancer cells have lost the normal control of the cell cycle and so divide out of control compared to normal cells. The sub-cellular machinery which controls the cell cycle is a complex biochemical device made up of a set of interacting proteins that induce and co-ordinate the essential processes of duplication and division of the contents of a cell. In the normal cell cycle, the control system is regulated such that it can stop at specific points in the cycle. The stopping points allow for systems of feedback control from the processes of duplication or division. They also provide points for regulation by environmental signals.

Gene expression plays an integral part in cell division and its control. Loss of control of cell division may in certain instances have its origin in an alteration in gene expression. Analysis of genetic alterations in cancer cells has revealed many genes which encode proteins involved in the control of cell division in some way.

Oncogenes are one family of such genes. Oncogenes are either expressed in cancer cells in a mutated form or they are over-expressed. The products of such oncogenes promote cell proliferation. The non-mutated or normally expressed version of an oncogene is known as a proto-oncogene and this is expressed in normal cells and encodes a constituent protein of the normal cellular machinery.

Another kind of gene product connected with cancer is that expressed by tumour-suppressor genes and the gene products serve to restrain cell proliferation. Mutation of a tumour-suppressor gene or loss of function of the gene product results in a loss of the normal control on proliferation and the cell divides out of control.

The study of cancer cells and their oncogenes or tumour-suppressor genes has helped to show how growth factors regulate cell proliferation in normal cells through a complex network of intracellular signalling cascades. These cascades ultimately regulate gene transcription and the assembly and activation of the cell cycle control system. As knowledge increases about the component parts of the cell cycle control machinery and how it operates, the possibilities for correcting the loss of control in cancer cells are increased. Essential points of control and essential proteins can be identified in the control hierarchy and potentially targetted with drugs to act as promoters or inhibitors, as required.

The cell cycle control system is based on two main families of proteins. The first is the family of cyclin-dependent protein kinases (CDK) of which there are a number of varieties, e.g. CDK 1 and CDK 2. CDK phosphorylates selected proteins at serine and threonine residues. The second sort of protein is a family of specialised activating proteins called cyclins that bind to CDK molecules and control their ability to phosphorylate targets. Cyclins themselves undergo a cycle of synthesis and degradation within each division of the cell cycle. There are a variety of species of cyclin, e.g. cyclin A and cyclin B.

Chao Y et al (1998) Cancer Research 58: 985-990 report a correlation between over-expression of cyclin A in patients and proliferative activity of tumour cells compared to those patients expressing a normal cyclin A level. Patients over-expressing cyclin A had a shorter medium disease-free survival time than those who did not over-express. Chao et al (1998) also report that a cyclin A-interacting protein (Skp 2) did not exhibit the same correlation with tumour cell activity as cyclin A when over-expressed. Chao et al (1998) remark on how expression of Skp 2 appears to be involved in the control of cell cycle progression but caution that the actual biochemical function of Skp 2 is still not known.

In a more recent paper, Chao Y et al (1999) Cancer Letters 139: 1-6 conclude that cyclin A may provide a useful target for the exploration of new anti-hepatocelluar carcinoma (HCC) therapeutics. In particular, Chao et al (1999) showed that an over-expression of cyclin A in HCC cells could be inhibited with antisense mRNA for the cyclin A gene. Although an over-expression of Skp 2 is apparently also associated with HCC cell proliferation, Chao et al (1999) indicate that the biochemical function of Skp 2 remains unknown. For example, the results of an experiment seeking to block over-expression of Skp 2 using antisense mRNA suggests that abnormal Skp 2 expression has no direct correlation with HCC proliferation.

The activity of CDK is subject to regulation in the cell and a CDK inhibitor protein (p27) has been identified. In normal cells p27 has been shown to regulate the action of CDK's that are necessary for DNA replication. Levels of p27 are found to be high in quiescent cells and low in cells stimulated to divide. p27 appears to act as a brake on cell division by inhibiting activated CDK which itself drives cells to divide. A reduction in the level of p27 frees activated CDK from inhibition and drives cells to divide. Consistent with this activity of p27 is the way in which its destabilisation correlates generally with tumour aggressiveness and poor prognosis for cancer patients.

The cell cycle control system is a dynamic system and p27 itself does not remain at a constant level in the cell. The level is different depending on the point in the cell cycle. Lower levels of p27 arise due to breakdown via ubiquitination and subsequent proteasome-mediated degradation. A requirement for ubiquitin-mediated degradation of p27 is phosphorylation of the threonine residue 187 (T187) by activated CDK. The enzymes needed for ubiquitination of phosphorylated p27 are not known, although from knowledge of ubiquitination in systems such as yeast it is expected that there may be a human ubiquitin-protein ligase (E3) specific for p27.

Sutterlüty H et al (1999) Nature Cell Biology 1: 207-214 report that Skp 2 promotes the degradation of p27 in cells via the ubiquitination pathway. Skp 2 is a protein member of the F-Box-Protein (FBP) family. Skp 2 appears to be a p27 specific receptor of an Skp 1, CuIA (Cdc53), F-Box Protein (SCF) complex. Such complexes are known in yeast and act as ubiquitin-protein ligases (E3) in which the FBP subunit has specificity for the substrate for ubiquitination. E3 facilitates the transfer of an activated ubiquitin molecule from a ubiquitin-conjugating enzyme (E2) to the substrate to be degraded. Similarly, in humans there are SCF complexes and Skp 2 is an FBP which has an ability to interact specifically with p27 and which appears to be essential in the ubiquitin-mediated degradation of p27. Both in vivo and in vitro, Skp 2 is found to be a rate-limiting component of the cellular machinery which ubiquitinates and degrades phosphorylated p27.

Skp 2 appears to be the product of a single gene and as such has an unusual ability in that it is able to drive cells to divide. This ability is shared with only a few other known gene products, e.g. E2F-1, c-Myc and cyclin E-CDK2 complexes. Timely accumulation of Skp 2 at the G1/S transition of the cell cycle may be one of the few rate-limiting steps controlling the initiation of DNA replication in mammalian cells. Sutterlüty et al (1999) found that a mutant of Skp 2 which does not assemble into an SCF complex was defective in promoting the elimination of ectopically produced wild-type p27. Also, mutant Skp 2 produced an activation of cyclin-E/A associated kinases and an induction of the S phase. Skp 2 also appears to have an independent binding site for CDK and activated CDK is involved in the phosphorylation of the T187 residue of p27. Sutterlüty et al (1999) also note how normal Skp 2 induces an accumulation of cyclin A protein, even when activation of cyclin-E/A-dependent kinases and entry into S phase are blocked by the expression of a non-degradable p27 mutant. What is concluded is that Skp 2 up-regulates cyclin A and independently of this down-regulates p27. The mechanism by which Skp 2 up-regulates cyclin A is not known. There is a suggestion that observed increased levels of Skp 2 in transformed cells might contribute to the process of tumourigenesis, at least partly, by causing an increase in the rate of degradation of the tumour suppressing agent p27. A lack of p27 expression correlates with a reduced disease-free survival of patients with colorectal and breast cancer. Also, p27 has been found to be haplo-insufficient for tumour suppression.

Carrano A. C. et al (1999) Nature Cell Biology 1: 193-199 report how Skp 2 interacts physically with phosphorylated p27 both in vitro and in vivo. Whilst every component of the ligase machinery required for p27 ubiquitination remains to be discovered, Carrano et al (1999) demonstrate that Skp 2 is a critical part of this machinery and provides substrate recognition and specificity for p27. Antisense oligonucleotides against Skp 2 were found to decrease Skp 2 expression in cells and thereby result in increased levels of endogenous p27. Carrano et al (1999) also confirm an additional need for cyclin E-CDK 2 or cyclin A-CDK 2 for ubiquitination of p27 to take place. p27 degradation in cells appears to be subject to dual control by accumulation of both Skp 2 and cyclins following mitogenic stimulation.

Of interest to scientists elucidating the molecular bases of cancer is a field of study relating to the molecular basis of the control of gene expression. Previously unconnected with the apparently essential roles of Skp 2 and p27 in cancer is the protein Pontin 52 reported in Bauer A. et al (1998) Proc. Natl. Acad. Sci. USA 95: 14787-14792. Pontin 52 is a nuclear protein which has a binding site for the TATA box binding protein (TBP). Pontin 52 also has a binding site for β-catenin. Pontin 52 is a ubiquitous and highly conserved ATP-dependent helicase protein. β-catenin is normally a cytoplasmic protein which has one role of providing a cytoplasmic anchor for other molecules involved in intercellular connections. β-catenin is also known to be a participant in the Wnt signalling pathway. In the Wnt signalling pathway, β-catenin becomes stabilised in the cytoplasm and can therefore interact with transcription factors of the lymphocyte enhancer factor-1/T-cell factor (LEF-1/TCF) family. Interaction with these transcription factors causes β-catenin to become localised in the nucleus. Binding of β-catenin with Pontin 52 provides the necessary molecular bridge between β-catenin and the TBP. The TBP binds to DNA, particularly in the TATA box region of gene promoters.

A protein equivalent to Pontin 52 is found in rats and is called TIP49. Wood M. A. et al (2000) Molecular Cell 5: 321-330 observe that c-Myc oncogenic transformation of cultured rat embryo fibroblasts required TIP49 as an essential co-factor. TIP49 was found to complex with c-Myc in vivo. TIP49 is a highly conserved protein and has ATPase and DNA helicase activity. Another similar co-factor protein, TIP48, also appears essential. In the present specification reference to either TIP48 or TIP49 are to be construed as references to the relevant proteins in humans or in any animal species (e.g., Pontin 52).

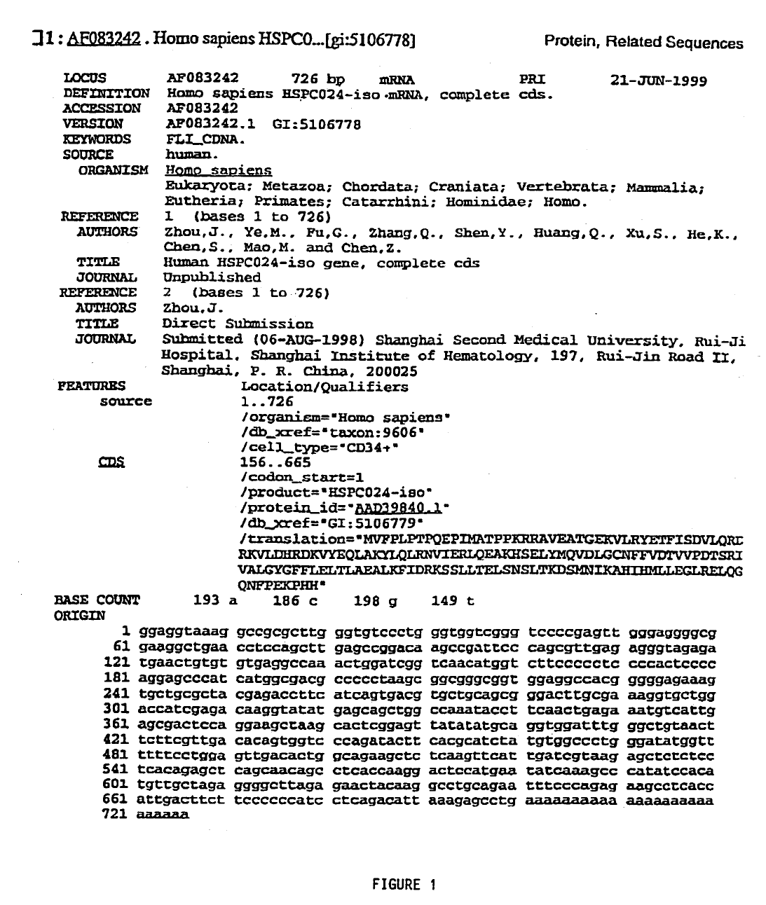

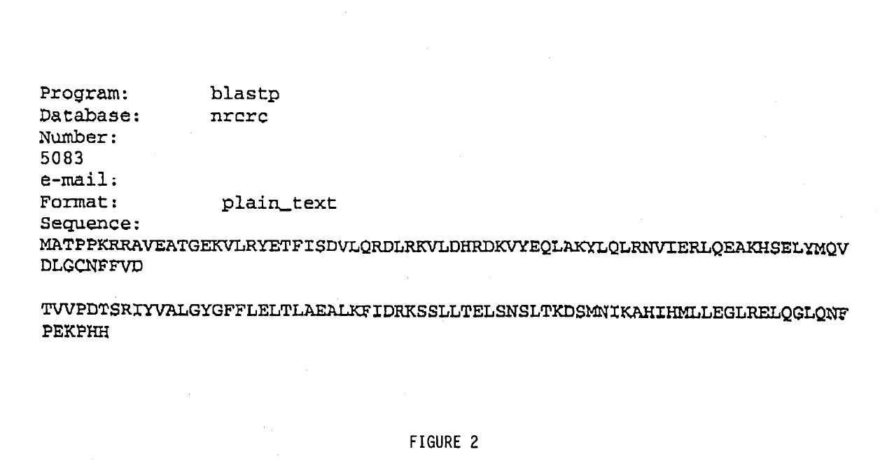

Genebank sequence AF083242 comprises 726 base pairs and is shown in FIG. 1 as SEQ ID NO:2. The DNA sequence is not known to encode any known structural or functional protein, nor is the sequence known to have any regulatory or other effects on the genome. Also available is an amino acid sequence derived from the cDNA sequence. This is set forth as SEQ ID NO:1 in FIG. 2.

There are also a number of expressed sequence tags (ESTs) having varying degrees of homology with the Genebank sequence (SEQ ID NO:2 or SEQ ID NO:3) shown in FIG. 1. However, none of the ESTs are correlated to any known gene or gene product.

The inventors have screened a variety of different cancer cell types for levels of expressed Skp 2 and p27. The inventors have also carried out co-transformation of primary rodent fibroblasts with both Skp 2 and H-RAS^{G12V}. Out of these experiments the inventors have discovered that Skp 2 is an oncogene responsible for many human cancers.

In exploring the oncogenic function of Skp 2 the inventors have unexpectedly discovered a novel protein called Skp 2-associated protein one (STAP1). The inventors generated antibodies against STAP1 and used these antibodies to immunoprecipitate STAP1 from HeLa cells. The immunoprecipitates were surprisingly found to contain several STAP1-co-immunoprecipitating proteins. The proteins including STAP1 were found to form a complex. The molecular weights of proteins were determined by mass spectrometry and then databases of proteins and gene sequences were searched to try and identify the proteins. Quite unexpectedly the STAP1-containing complex of proteins is found to include TIP48, TIP49, RPB 5 (RNA pol II subunit 5), RMP1 (RNA pol II mediator protein), prefoldin, as well as other hitherto unknown proteins.

Without wishing to be bound by any particular theory, the inventors have realised that Skp 2 represents an oncogene which can interact through STAP1 and its complex with known elements of a transcriptional control apparatus, in particular TIP49 (and TIP48) and that this link provides a new point of attack for inhibitors of protein-protein binding and enzymic activities. Such inhibitors are expected to have anti-proliferative and therefore anti-cancer properties. In the light of these discoveries, suitable screening assays can now be developed to identify new anti-cancer agents.

In one aspect the invention therefore provides an Skp 2 binding protein (STAP1) having a molecular weight of about 18 kD or less, or a polypeptide fragment thereof. Measurement of molecular weight is preferably performed using standard denaturing electrophoresis on Laemili SDS-PAGE.

The invention also provides an Skp 2 binding protein (STAP1) or polypeptide fragment thereof comprising an amino acid sequence substantially as set forth in FIG. 2 (SEQ ID NO: 1), or a sequence substantially homologous therewith, particularly a degree of identity (homology) of at least 60%. The Skp 2 binding protein preferably has a molecular weight of about 18 kD or less.

The amino acid sequence identity is at least 70%, preferably at least 80%, more preferably at least 90%, even more preferably 95%, most preferably at least 99%.

An Skp 2 binding protein (STAP1) of the invention, or polypeptide fragment thereof, is encoded by a nucleic acid sequence substantially as set forth in FIG. 1 (SEQ ID NO:2), SEQ ID NO:3 or 4, or a sequence having at least 70% homology (identity) therewith, that is capable of hybridizing under low stringency conditions thereto and encoding an Skp 2 binding protein or fragment thereof. Low stringency conditions employs around 0.01×SSC buffer compared to high stringency which employs about a 10 fold greater concentration. The sequence homology may be at least 80%, preferably at least 90%, more preferably at least 95%, even more preferably 97.5%, most preferably at least 99%.

In accordance with the invention another nucleotide sequence encoding STAP 1 is set forth in SEQ ID NO:4. All references to SEQ ID NOS: 2 and 3 are to be construed as also including reference to SEQ ID NO:4 as an alternative.

The STAP1 protein of the invention and its variants have binding affinity for, and/or association affinity with, at least one transcription regulatory factor, optionally other proteins or polypeptides of the cell. Also included are polypeptide fragments of the STAP1 proteins.

Preferred STAP1 proteins have binding affinity for, and/or association affinity with, one or more of TIP48 (EP 09261 5A1), TIP48 (EP 09261 5A1), prefoldin, RPB5 (Cheong et al., 1995, EMBO J. 14:143-150) and RMP1 (Dorjsuren et al., 1998, Mol. Cell. Biol. 18:7546-7555), optionally other proteins or polypeptides. Polypeptide fragments of these STAP1 proteins are part of the invention.

Variants of the STAP1 protein and polypeptides are also of utility and so the invention includes all forms of mutant variants, for example wherein at least one amino acid is deleted or substituted. The Skp 2 binding region is formed by amino acid residues 66 to 119 and so in preferred variants there is no change in sequence in this region compared to the native sequence.

Thus, any deletion or substitution is preferably other than in the Skp 2 binding site region of amino acids 66 to 119.

Any changes involving substitution of amino acids are preferably neutral or conservative substitutions.

Other variants include proteins or polypeptides comprising at least one additional amino acid in the sequence, and/or further comprising an additional amino acid sequence or domain; preferably any additional amino acid, sequence or domain being inserted into other than the Skp 2 binding region of amino acids 66 to 119.

Also provided by the invention is a polypeptide comprising just a functional Skp 2-binding region.

Further variants of the STAP1 proteins or polypeptides include those wherein at least one of the amino acids in the sequence is a natural or unnatural analogue. Also, one or more amino acids in the sequence may be chemically modified, e.g. to increase physical stability or to lower susceptibility to enzymic, particularly protease or kinase, activity.

In another aspect the invention provides a nucleic acid antisense to all or a part of a nucleic acid of SEQ ID NO:2 or SEQ ID NO:3, or antisense to a sequence having at least 70% homology with SEQ ID NO:2 or SEQ ID NO:3, that is capable of hybridizing under low stringency conditions thereto, and which encodes a STAP1 protein or polypeptide as hereinbefore described. The sequence to which the nucleic acid is antisense to may have at least 80% homology with SEQ ID NO:2 or SEQ ID NO:3, preferably at least 90%, more preferably at least 95%, even more preferably at least 95%, most preferably at least 99%.

Antisense nucleic acids are preferably to be at least 10 bases long, more preferably at least 15 even more preferably at least 50 bases long.

In certain embodiments at least some of the nucleotide residues of the antisense sequence may be made resistant to nuclease degradation and these can be selected from residues such as phophorothioates and/or methylphosphonates.

The antisense nucleic acids as hereinbefore described can advantageously be used as pharmaceuticals, preferred pharmaceutical applications being for the manufacture of a medicament for the prophylaxis or treatment of cancer. Without wishing to be bound to any particular theory, the inventors believe that an antisense inhibition of STAP1 expression in cancer cells may reduce the level of the transcription regulatory complex containing STAP1. This is turn may switch off genes involved in proliferation.

The invention also provides a nucleic acid of SEQ ID NO:2 or SEQ ID NO:3, or a sequence of at least 70% homology thereto, that is capable of hybridizing under low stringency conditions, and which encodes a STAP1 protein or polypeptide of the invention for use as a pharmaceutical. The sequence homology with SEQ ID No:2 may be at least 80%, preferably at least 90%, more preferably at least 95%, even more preferably 97.5%, most preferably at least 99%. Some of the nucleotide residues may be made resistant to nuclease degradation and selected from phophorothioates and/or methylphosphonates, for example. The nucleic acids are of particular value for the manufacture of a medicament for the prophylaxis or treatment of cancer. Not wishing to be bound by any particular theory the inventors believe that an anti-cancer activity of the nucleic acids may be through a mechanism of sense suppression.

Thus, the invention also provides a method of preventing or treating cancer comprising administering to an individual an effective amount of a nucleic acid antisense to all or a part of a nucleic acid of SEQ ID NO:2 or SEQ ID NO:3, or antisense to a sequence having at least 70% homology with SEQ ID NO:2 or SEQ ID NO:3, that is capable of hybridizing under low stringency conditions thereto, and which encodes a STAP1 protein or polypeptide as hereinbefore described.

The invention also provides a method of preventing or treating cancer comprising administering to an individual an effective amount of a nucleic acid of SEQ ID NO:2 or SEQ ID NO:3, or a sequence of at least 70% homology thereto, that is a sequence capable of hybridizing under low stringency conditions thereto, and which encodes a protein or polypeptide as hereinbefore described.

Other embodiments of the invention include nucleic acid constructs comprising (a) at least one nucleic acid sequence portion encoding a STAP1 protein or polypeptide as hereinbefore described or a sequence at least 70% homologous thereto, (b) antisense nucleic acids as hereinbefore described, or (c) nucleic acids as hereinbefore described and at least one nucleic acid sequence not encoding a STAP1 protein or polypeptide of the invention. Such constructs are not naturally occurring sequences. The constructs are artificial but they do lack essential sequences of DNA which might permit them to function as vectors. They may include nucleic acid sequences that function as linkers or restriction sites. Preferred constructs are synthesised using methods of oligonucleotides synthesis well known to those of skill in the art.

Also provided are vectors comprising (a) a nucleic acid sequence portion encoding a STAP1 protein or polypeptide as hereinbefore described or a sequence at least 70% homologous thereto, (b) an antisense nucleic acid as hereinbefore described, (c) a nucleic acid as hereinbefore described, or (d) a construct as hereinbefore described.

Preferred vectors are expression vectors, preferably plasmids or viruses although cloning vectors are also provided for, optionally in the form of plasmids.

The invention provides host cells containing vectors; preferably the host cell expresses a STAP1 protein or polypeptide of the invention. Preferred host cells are eukaryotic cells, more preferably insect cells or mammalian cells.

Constructs, vectors and transformed host cells of the invention are of use as pharmaceuticals particularly as a medicament for the prophylaxis or treatment of cancer.

The invention further provides antibodies reactive against a STAP1 protein or polypeptide of the invention, preferably the antibodies are specifically reactive against the STAP1 protein or polypeptide. The antibodies may be monoclonal or polyclonal and other forms e.g. humanised are possible within the scope of the invention. Antibodies are also of use as pharmaceuticals.

The invention provides a method of preventing or treating cancer comprising administering to an individual an effective amount of a construct, vector, host cell or antibody of the invention.

In another aspect the invention also provides a method of identifying anti-cancer compounds comprising measuring the binding of a test compound to a STAP1 protein or polypeptide of the invention, optionally also comprising measuring the binding of a control compound to a STAP1 protein or polypeptide.

In another method for identifying anti-cancer compounds there is measurement of the binding of a STAP1 protein or polypeptide of the invention to Skp 2, or a fragment or variant of Skp 2, in the presence of a test compound, optionally including measurement of the binding in the absence of a test compound.

Preferred methods are solid phase assays and in preferred embodiments the STAP protein or polypeptide of the invention, or the Skp 2 protein, fragment or variant thereof are immobilised to a substrate. Most preferred substrates are nickel or nickel coated, e.g. nickel coated microtiter plates. Either the STAP1 protein (or polypeptide) of the invention or Skp 2 (including Skp 2 fragments or Skp 2 variants) are labelled.

The label may be a fluorescent label, an enzyme label, biotin, a metal sol particle or a radiolabel. In preferred embodiments the label is europium.

Alternatively, the methods of screening for anti-cancer agents may be liquid phase assays, preferably employing fluorescent labelling of each of the STAP1 protein or polypeptide of the invention and Skp 2 (also Skp 2 fragments or Skp 2 variants), i.e. dual labelling.

The invention therefore includes the use of Skp 2, an Skp 2 fragment, or an Skp 2 variant or fragment, in methods of identifying anti-cancer agents as hereinbefore described.

The invention also includes the use of a STAP1 protein or polypeptide as hereinbefore described in a method of identifying anti-cancer agents as hereinbefore described.

An antisense nucleic acid of the invention can also be used as a probe for determining expression of a STAP1 protein in a cell. This may be of practical utility in circumstances where host cells have been transfected with the STAP1 gene and it is desired to check for transcription of the gene. Also the antisense nucleic acid can be used as a research tool to identify transcription levels of the STAP1 gene in cancer cell samples.

The nucleic acid of SEQ ID NO:2 or SEQ ID NO:3, or a sequence of at least 70%, preferably 80%, more preferably 90%, even more preferably 95%, most preferably 99% homology thereto, ideally fragments thereof, can be used as a primer or a probe for nucleic acid encoding a STAP1 protein or polypeptide.

Nucleic acid primers may be of use in performing PCR amplification of samples of nucleic acids encoding STAP1. PCR can be used as an analytical tool, optionally in conjunction with nucleic acid probes specific for STAP1, for detection of the STAP1 gene and/or its expression.

In another aspect, the invention provides a complex comprising a STAP1 protein or polypeptide as hereinbefore described and one or more other proteins or polypeptides. In preferred complexes at least one of the other proteins or polypeptides has ATPase activity. In other preferred complexes at least one of the other proteins or polypeptides has DNA helicase activity.

Particularly preferred complexes comprise one or more of TIP48, TIP49, prefoldin, RPB 5 and RMP 1, optionally one or more further proteins or polypeptides. The subunits STAP1, TIP48, TIP49, RPB 5, RMP 1 may be present in a ratio of about 1:1:1:1:1, although other ratios are possible. Optionally, the additional proteins or polypeptides may also be in a stoichiometric ratio of 1:1, but again other ratios are possible.

The invention also provides a transcription regulatory protein complex comprising TIP48 and/or TIP49 and three or more other proteins or polypeptides. These other proteins or polypeptides may be as hereinbefore described.

In any of the complexes of the invention hereinbefore described the constituent protein or polypeptide subunits may each have a molecular weight in the range 5 to 500 kD, preferably 5 to 300 kD, more preferably, 10 to 200 kD, even more preferably 10 to 100 kD. SDS-PAGE or mass spectrometry provide ways of establishing molecular weights.

Complexes of the invention as hereinbefore described may be obtainable by immunoprecipitation using an antibody reactive against STAP1 or polypeptide fragments of STAP1. Ideally, complexes of the invention are substantially free of other cellular contaminants. Thus, isolated complexes may be of at least 80% purity, preferably 90% purity, more preferably 95% purity, even more preferably 99% purity. Purity can be determined by various methods, e.g. SDS-PAGE.

Alternative ways of producing complexes of the invention may be to assemble them from constituent protein or polypeptide subunits. One way is to have a cell transformed to overexpress each of the constituent subunits so that assembly of the complex takes place in the cell. A preferred expression system employs transformed insect cells.

Another way is to mix the constituent subunits together in vitro under conditions sufficient for self assembly of the complex. Preferably, the mixing of subunits occurs substantially simultaneously. There are many other possibilities of mixing including assembly of partial complexes in transformed cells followed by isolating and mixing them with the remaining subunits in vitro under conditions promoting self assembly of the whole complex. Also, partial complexes can be made in vitro by mixing and then mixed with the remaining subunits. The order of mixing subunits or partial complexes in vitro is not believed to be critical in order to yield complexes.

In another aspect the invention provides a method of identifying an anti-cancer agent comprising contacting an amount of a complex as hereinbefore described with a test compound and then determining one or more of: (a) the amount of intact complex remaining, (b) the amount of intact complex lost, or (c) the amount(s) of free protein or polypeptide subunit(s) released from the complex.

The amount of complex may be determined by measuring one or more activities of the complex, preferably an enzymic and/or ligand binding activity. Where ligand binding is measured then the ligand may be selected from a nucleic acid or a protein, preferably the protein binding activity is an oncogene product. e.g. c-Myc or Skp 2 binding activity, beta-catenin binding activity, Hbx binding activity or RNA polymerase II binding activity. If an enzymic activity is measured then it may be ATPase activity, and/or DNA helicase activity.

In methods which determine the amount(s) of free protein or polypeptide subunits lost from the complex then the free protein or polypeptide subunit(s) may be one or more of RBP 5, RMP 1, prefoldin, TIP48, TIP49 or a STAP1 protein or polypeptide as hereinbefore described. Free protein or polypeptide subunit amounts may be determined by measuring an enzymic and/or ligand binding activity. When a ligand binding assay then the ligand may be selected from a nucleic acid or a protein, preferably the protein binding activity is an oncogene product e.g. c-Myc or Skp 2 binding activity, beta-catenin binding activity, Hbx binding activity or RNA polymerase II binding activity. When an enzymic activity is determined then it may be selected from one or more of ATPase activity or DNA helicase activity.

In all methods of anti-cancer agent screening there may be the further step of forming the complex from its protein subunit components prior to contact with the test compound.

In another aspect the invention provides a method for identifying an anti-cancer agent, but not an agent against cancers mediated by c-Myc, comprising contacting a test compound with TIP49 and/or TIP48 and measuring ATPase activity, optionally also measuring ATPase activity in the absence of a test compound.

In an alternative aspect the invention provides a method for identifying an agent active against cancer cells expressing Skp 2 comprising contacting TIP49 and/or TIP48 with a test compound and measuring ATPase activity, optionally also measuring ATPase activity in the absence of a test compound.

Preferred active agents which the method is able to identify are those active against cancer cells which overexpress Skp 2. Other preferred agents identified by the method of the invention are those active against cancers which are not mediated by c-Myc.

Another aspect of the invention is the use of one or more proteins selected from TIP48, TIP49, RPB5, RMP1 or a STAP1 protein or polypeptide as hereinbefore described in a method of screening for anti-cancer agents, preferably any of the methods hereinbefore described. Allied to this aspect of the invention is the use of any one or more of TIP48, TIP49, RPB 5, RMP 1 or a STAP1 protein or polypeptide of the invention for in vitro assembly of a complex as hereinbefore described.

The invention permits the identification of anti-cancer agents by performance of any of the methods of screening described herein. Preferred anti-cancer agents are those which inhibit proliferation of the cancer cells and which may be general anti-proliferative agents. The invention includes all agents identified by performing the methods, including antisense nucleic acids and antibodies, and the use of these agents as pharmaceuticals, particularly as medicaments for the prophylaxis or treatment of cancer.

The invention includes a method of preventing or treating cancer comprising administering to an individual an effective amount of a compound identified by a screening method of the invention described above.

Preferred embodiments of the invention will now be described by way of example and where convenient with reference to drawings in which:

FIG. 1 shows a nucleotide sequence of STAP1 (SEQ ID NO:2).

FIG. 2 shows a derived amino acid sequence of STAP1 (SEQ ID NO:1).

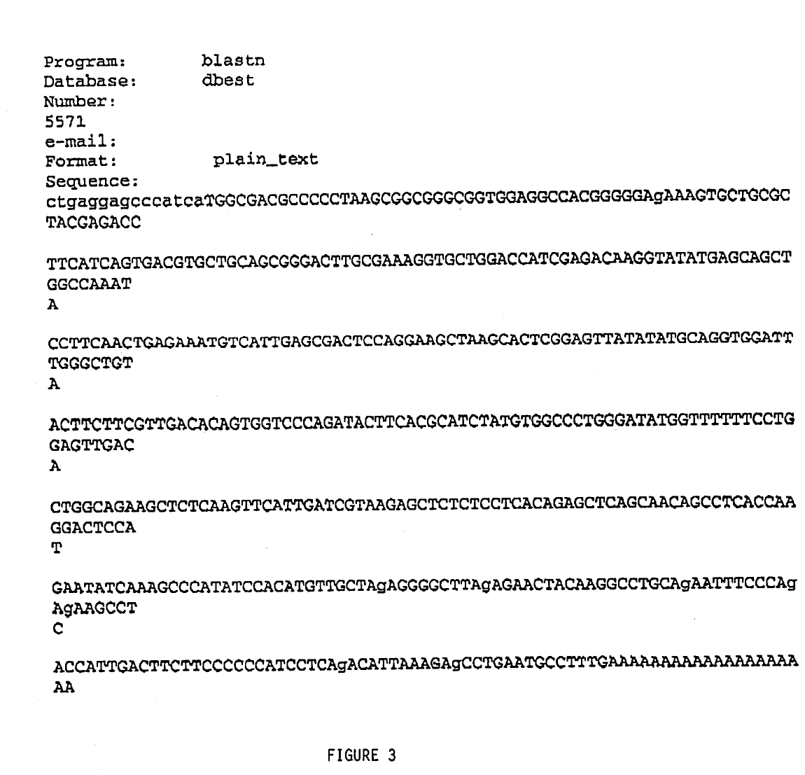

FIG. 3 shows another nucleotide sequence of STAP1 (SEQ ID NO:3).

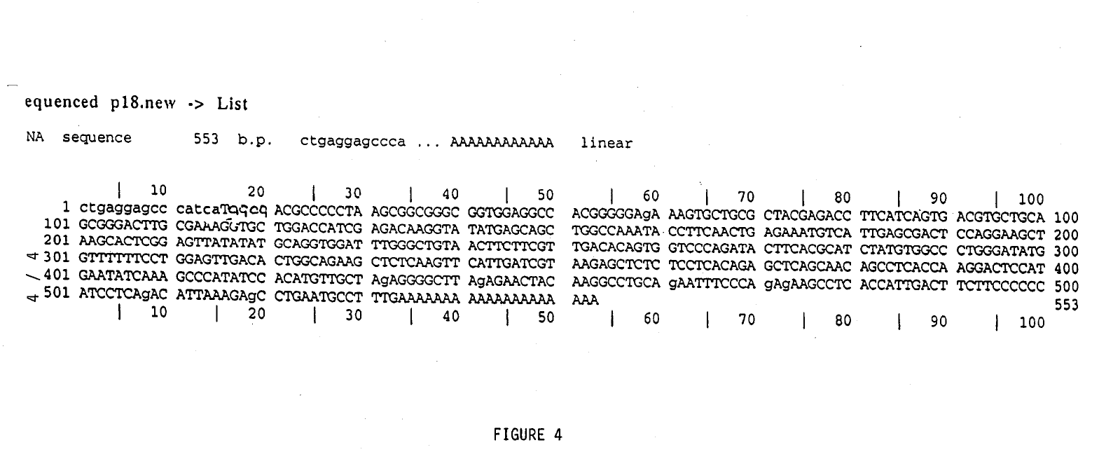

FIG. 4 shows another nucleotide sequence of STAP1 (SEQ ID NO: 4)

### EXAMPLE 1

### Skp 2 and H-Ras^{(G12V)} Transfection of Cells Transforms Them

Skp 2 co-operates with H-Ras^{G12V} to cause cellular transformation of primary rodent fibroblasts as scored by colony formation in soft agar and tumour formation in nude mice. Such transformants express significantly lower levels of p27 than normal fibroblasts or E1A/H-Ras^{G12V}-transformed derivatives.

A sensitive assay of functional properties of candidate oncogenes derives from the use of embryo cell cultures that can be transfected with these genes singly or in combination. When introduced into rat embryo fibroblasts, oncogenes such as E1A or E2F1 are able to transform them only in the presence of a co-introduced, collaborating oncogene like the oncogenic version of H-Ras in which Gly¹² was changed to Val (G12V). Mammalian expression plasmids encoding Skp 2 and H-Ras^{G12V} were transfected either alone or in combination into primary rat embryo fibroblasts (REFs). After selection in G418 for 3 weeks, plates were scored for the presence of morphologically transformed colonies. In the absence of H-Ras^{G12V}, Skp 2 alone failed to give rise to morphologically transformed foci. In contrast, addition to H-Ras^{G12V} together with the Skp 2 gene gave rise to substantially increased number of morphologically transformed colonies, ranging on average from 70-110 colonies pre plate. Colonies produced by transfection of Skp 2 and H-Ras^{G12V} were easily established and gave rise to cell lines that grew rapidly in culture. These Skp 2/H-Ras^{G12V}-expressing cells were plated into semisolid medium (fresh medium containing 0.3% agar). After 2 weeks plates were analysed for the presence of colonies. Skp 2/H-Ras^{G12V}-expressing cells readily formed colonies in soft agar, which is a strong criterion for cultured cell transformation. In addition, 1×10⁶ Skp 2/H-RaS^{G12V}-expressing cells were injected in the flank of 2-3 week old nude mice. Mice were scored for the presence of tumours at the injection site. At two weeks thereafter, tumour formation was detected in all experimental animals injected with Skp 2/H-Ras^{G12V}-expressing cells but not with control REFs. The results of the cotransfection experiments shows that Skp 2 can act as an oncogene.

### EXAMPLE 2

### Immunohistochemical Analysis of Cells Shows a Significant Inverse Relationship Between the Levels of Skp 2 and p27 in Tumour Cells

Skp 2 expression was analysed in a series of human primary oral squamous cell carcinomas, breast carcinomas, lymphomas and prostate cancers. In general, 5 micrometer thick formalin fixed and paraffin embedded tissue sections were stained for p27 and Skp 2 protein by immunohistochemistry using a monoclonal antibody against p27 and polyclonal antibody against Skp 2.

Monoclonal antibodies against p27 are available from Transduction Laboratories. Polyclonal antibodies against Skp 2 are readily raised by persons of average skill in the art by immunisation of an animal with a suitably purified Skp 2 preparation. The polyclonal antibodies can additionally be affinity purified as described by Lisztwag J et al (1998) EMBOJ 17: 368-363.

The results showed that the expression of p27 and Skp 2 is inversely related in all cancers tested. This confirms that Skp 2 is most likely to function as an oncogene.

These results implicate a substrate-recognition subunit of an SCF ubiquitin protein ligase complex in the development of human cancer.

### EXAMPLE 3

### Isolation and Cloning of a cDNA Encoding an Skp 2 Associated Protein (STAP1)

A yeast-two hybrid screen was performed using Skp 2 as a bait. From this a cDNA was cloned that encodes for a protein of about 18 kDa that we now refer to as STAP1 (for Skp 2-associated protein one). The STAP1 protein is hitherto unknown.

About 1×10⁶ clones were screened from a HeLa cell library constructed in pGAD-GH (Clontech) which baits encoding residues 101-423 of human Skp 2 cloned in the GAL4 DNA-binding domain vector pAS2-1. Interacting clones were identified after selection on triple-dropout media (minus Leu/Trp/His with 25 mM 3-amino-triazole), and assaying for strong-galactosidase activity. 35 positive clones were sequenced. Sequence comparison revealed that all cloned cDNAs encode for the novel protein STAP1, having a molecular weight of about 18 kD.

### EXAMPLE 4

### Production of Recombinant STAP1

Human STAP1 full-length version was expressed in Escherichia coli BL21 as glutathione-S-transferase (GST) fusion proteins and purified on glutathione-sepharose, eluted with glutathione. Methodology is described in Kaelin et al (1991) Cell. 64: 521-532 and also Krek et al(1994) Cell. 78: 161-172.

### EXAMPLE 5

### Preparation of Antibodies Reactive Against STAP1

Eluted STAP1 material from example 4 above was injected into mice to generate monoclonal antibodies. A routine monoclonal antibody production protocol was undertaken as will be well known to those of skill in the art. Polyclonal antiserum and antibodies against STAP1 were also generated by injection of the STAP1 eluted material of example 4 above into rabbits following a standard form of protocol which will be familiar to those of skill in the art.

### EXAMPLE 6

### Immunoprecipitation and Electrophoretic Separation of a Complex Containing STAP1 from HeLa Cells

Large scale immunoprecipitation was carried out with HeLa whole cell extracts. 100 μg of monoclonal anti-STAP1 antibody coupled to protein A was added to 50 ml of HeLa nuclear extracts (from about 2 to 10⁹) and rotated for 2 hr at ₄° C. The immunoprecipitates were then washed in 25 ml of TNN [20 mM Tris-HCl (pH 8.0), 0.1 M NaCl, 1 mM EDTA, 0.5% NP-40] four times. The precipitated proteins were eluted with 300 μl 0.2M Glycine (pH 2.5) into Laemmli buffer and separated on a 10% SDS-polyacrylamide gel. The gel was then stained with silver.

### EXAMPLE 7

### Analysis of STAP1-Associated Protein by Mass Spectrometry

The SDS-PAGE separated proteins were excised from the gel of example 6, reduced with DTT, alkylated with iodoacetamide and cleaved with trypsin (Promega, sequencing grade) as described by Shevchenko, A., Wilm, M., Vorm, O. and Mann, M. (1996) Anal. Chem., 68: 850-858. The extracted tryptic peptides were desalted with 5% formic acid, 5% Methanol in H₂O on a 1 μl Poros P20 column and concentrated to 1 μl with 5% formic acid, 50% Methanol in H₂O directly into the Nanoelectrospray ionisation (NanoESI) needle. NanoESI mass spectrometry (MS) was performed according to the published method of Wilm, M. and Mann, M. (1996) Anal. Chem., 68: 1-8. The mass spectra was acquired on an API 300 mass spectrometer (PE Sciex, Toronto, Ontario, Canada) equipped with a NanoESI source (Protana, Odense, Denmark). See also W. R. Pearson & D. J. Lipman (1998) PNAS, 85: 2444-2448.

The STAP1-containing complex is found to contain a large number, about 20 or so proteins. As well as STAP1, the complex has also been found to comprise TIP48, TIP49 (two evolutionarily conserved ATPases and DNA helicases), RPB5 (RNA pol II subunit 5), RMP1 (RNA pol II mediator protein) and at least three other hitherto unknown proteins.

### EXAMPLE 8

### Analysis of the STAP-Containing Complex by Sucrose Density Gradient Centrifugation and Western Blotting

A crude HeLa cell extract was subjected to 5-30% and 10-30% (w/v) density centrifugation. The sample was loaded in TNN buffer made up of 10 mM Tris (pH 7.5), 250 mM Na Cl, 0.5% NP40, 1MM DTT, sodium vanadate, PMSF and aprotinin. The buffer was also used in the sucrose gradient but the NP40 was omitted.

Each of the fractions was mixed with sample buffer and subjected to standard Laemlli denaturing SDS-PAGE at 12%. A number of gels were run and then each was blotted with an antibody. Polyclonals against RMP1 and TIP49 were used, as were monoclonals against RPB5, TIP48, STAP1 and Skp 2. The lanes of the blotted gels are aligned with their respective sucrose fractions and what is apparent is that the components of the STAP1-containing complex are clearly associated together and do not form part of the main peak of protein in the gradient. The components of the complex are all found in earlier eluting fractions where higher molecular weight proteins sediment. Skp 2 has a different pattern in the gradient compared to the STAP1-containing complex, peaking just before the main peak of protein.

Also noted for the first time is how TIP49 antibodies recognise a doublet on SDS-PAGE. There is an immunologically related TIP49 variant of slightly higher molecular weight.

### EXAMPLE 9

### Screening for Anti-Cancer Agents Which are Inhibitors of a STAP1-Associated DNA Helicase Complex

Small molecule compounds that disrupt specific interactions between the components of a STAP1-containing TIP49, TIP48, RPB5, RMP1, STAP1 and Skp 2, for example are putative anti-cancer agents. The component proteins of the complex are expressed in Sf9 insect cells using recombinant baculoviruses. All possible combinations of pairwise interactions between subunits of the complex are constructed and used to screen synthetic and natural compounds. In practice, coinfection of insect cells followed by immunoprecipitation with the appropriate antibody provides the complex substrate used in the screening assays. Coimmunoprecipitation between two of the above-noted components indicates a direct interaction and hence a target for disruption of interaction by putative anti-cancer agents. For example, STAP1 and Skp 2 coimmunoprecipiate when coexpressed in this system and provide a binding pair suitable as the basis of a screening assay for synthetic or natural compounds which disrupt that binding in some way. To screen for small molecular compounds, recombinant hexahistidine-tagged STAP1 is purified from insect cells and immobilized to the surface of nickel-coated 96-well plates. Immobilized STAP1 is incubated with purified biotinylated Skp 2 and washed. Subsequently, europium-labelled streptavidin is added. Then, time-resolved fluorescence of europium is monitored in the absence of presence of synthetic chemical libraries and natural products.

### EXAMPLE 10

### Screening for Anti-Cancer Agents Which are Inhibitors of TIP48 and/or TIP49 ATPase Activity

Recombinant TIP48 and TIP49 are expressed in E. coli using experimental procedures as described in Makino Y et al (1999) J. Biol. Chem. 274: 15329-15335. Purification of recombinant TIP48 and TIP49, as well as assays for ATPase activity and DNA helicase activity are also as described in Makino Y et al (1999). The purified recombinant proteins are used to screen for natural products or synthetic compounds which interfere with the normal enzymic activities of TIP48 and/or TIP49.

The screening assay is conveniently carried out in microtiter plates. TIP48 and/or TIP49 proteins are placed in the wells and one or both of the enzyme assays are carried out in the presence or absence of compounds from natural or synthetic chemical libraries. Advantageously, an ATPase microassay format can be used as described in Henkel R D et al (1988) Anal. Biochem. 169: 312-318.

All references, referred to herein and priority application GB 0011439.7 filed May 12, 2000, are incorporated by reference as if referred to individually.

1. A complex, said complex comprising a Skp 2 binding protein (STAP1) having a molecular weight of about 18 kD or less, or a polypeptide fragment thereof, and one or more other proteins or polypeptides.

2. The complex of claim 1, wherein the Skp 2 binding protein (STAP1) or polypeptide fragment thereof comprises an amino acid sequence substantially as set forth in FIG. 2 (SEQ ID NO: 1), or a sequence having at least 60% identity therewith.

3. The complex of claim 2, wherein the Skp 2 binding protein (STAP1) or polypeptide fragment is encoded by a nucleic acid sequence substantially as set forth in FIG. 1 (SEQ ID NO:2 or SEQ ID NO:3), or a sequence having at least 70% homology therewith.

4. The complex of claim 3, wherein at least one amino acid of the Skp2 binding protein (STAP1) is substituted with a neutral or conservative amino acid.

5. A complex as claimed in any preceding claim, further comprising at least one additional amino acid in the Skp2 binding protein (STAP1) sequence, and/or further comprising an additional amino acid sequence or domain, preferably any additional amino acid, sequence or domain being inserted into other than the Skp 2 binding region of amino acids 66 to 119.

6. A complex as claimed in any preceding claim comprising one or more of TIP48, TIP49, RPB 5 and RMP 1.

7. A complex as claimed in claim 14, wherein the subunits STAP1, TIP48, TIP49, RPB 5, RMP 1 are present in a ratio of about 1:1:1:1:1.

8. A complex as claimed in any preceding claim obtainable by immunoprecipitation.

9. A complex as claimed in any preceding claim substantially free of other cellular contaminants.

10. A complex as claimed in any preceding claim obtained by assembly in vitro from constituent protein or polypeptide subunits.

11. A complex as claimed in any preceding claim, wherein assembly takes place in a cell transformed to overexpress the constituent subunits.

12. A method of identifying an anti-cancer agent comprising contacting an amount of a complex of any preceding claim with a test compound and then determining one or more of: (a) the amount of intact complex remaining, (b) the amount of intact complex lost, or (c) the amount(s) of free protein or polypeptide subunit(s) released from the complex.

13. A method as claimed in claim 12, wherein the amount of complex is determined by measuring one or more activities of the complex, preferably an enzymatic and/or ligand binding activity.

14. A method as claimed in claim 13, wherein the ligand is selected from a nucleic acid or a protein.

15. A method as claimed in claim 14, wherein the enzymatic activity is ATPase activity and/or DNA helicase activity.

16. A method as claimed in claim 12, wherein free protein or polypeptide subunit amounts are determined by measuring an enzymatic and/or ligand binding activity.

17. A method as claimed in any of claims 12 to 16, further comprising the step of forming the complex from its protein subunit components prior to contact with the test compound.

18. The use of RPB 5 and/or a Skp 2 binding protein (STAP 1) or fragment thereof in a method of screening for anti-cancer agents, preferably a method of any of claims 12-17.

19. The use of RPB 5 and/or a Skp 2 binding protein (STAP 1) or fragment thereof for assembly of a complex in vitro.

20. An anti-cancer agent identified by a method of any of claims 12 to 17, preferably an anti-proliferative agent.

21. The agent of claim 20, wherein said agent is an antisense nucleic acid.

22. The agent of claim 20, wherein said agent is an antibody.

23. A method of preventing or treating cancer comprising administering to an individual an effective amount of an agent identified by a method of any of claims 12 to 17.

24. A nucleic acid antisense to all or a part of a nucleic acid of SEQ ID NO:2, or antisense to a sequence having at least 70% homology and capable of hybridizing with SEQ ID NO:2, SEQ ID NO:3 or SEQ ID NO:4 under low stringency conditions.

25. An antisense nucleic acid as claimed in claim 24, wherein at least some of the nucleotide residues are resistant to nuclease degradation and selected from phophorothioates and/or methylphosphonates, for example.

26. A nucleic acid of SEQ ID NO:2, SEQ ID NO:3 or SEQ ID NO:4, a sequence of at least 70% homology thereto, a fragment thereof or their complement for use as a pharmaceutical.

27. A method of preventing or treating cancer comprising administering to an individual an effective amount of a nucleic acid antisense to all or a part of a nucleic acid of SEQ ID NO:2 or SEQ ID NO:3, or antisense to a sequence having at least 70% homology with SEQ ID NO:2 or SEQ ID NO:3 and capable of hybridising under low stringency conditions thereto.

28. An antibody that specifically recognizes a SKP2 binding protein (STAP1) or fragment thereof, optionally monoclonal antibodies.

29. A method of identifying an anti-cancer agent comprising detecting binding or a change in binding of a SKP2 binding protein (STAP 1) or polypeptide fragment thereof in the presence of a test compound compared to when the test compound is absent, optionally also comprising measuring the binding of a control compound or protein.

30. A method as claimed in claim 29 being a solid phase assay, preferably wherein the SKP2 binding protein (STAP1) or polypeptide fragment or variant thereof is immobilised to a substrate, more preferably wherein the substrate is nickel or nickel coated.

31. A method as claimed in claim 29 or claim 30, wherein the SKP2 binding protein (STAP1), polypeptide fragment or variant thereof is labelled.

32. A method as claimed in claim 31, wherein the label is selected from a fluorescent label, an enzyme label, biotin, a metal sol particle or a radiolabel.
